(19) United States
(12) Patent Application Publication
(10) Pub. No.: US 2010/0022444 A1
(43) Pub. Date: Jan. 28, 2010

(54) COMBINATION THERAPY FOR MODULATION OF ACTIVITY AND DEVELOPMENT OF CELLS AND TISSUES

(75) Inventors: Patricia Kane, Millville, NJ (US); Edward Kane, Millville, NJ (US)

Correspondence Address: LAW OFFICES OF KHALILIAN SIRA, LLC, 9100 PERSIMMON TREE ROAD, POTOMAC, MD, 20854, US

(73) Assignee: BodyBio, Inc., Millville, NJ (US)

(21) Appl. No.: 12/509,160

(22) Filed: Jul. 24, 2009

Related U.S. Application Data
(60) Provisional application No. 61/135,852, filed on Jul. 24, 2008.

(51) Int. Cl. A61K 38/18; A61P 25/28; A61P 25/16

(52) U.S. Cl. 514/8

(57) ABSTRACT

The invention as disclosed herein provides methods and compositions for modulation of development and activities of cells and tissues in a subject in need of treatment thereof. The inventive methods and compositions involve the use of a combination therapy that comprises at least two active compositions, the first active composition comprising a balanced PC and an essential fatty acid composition and the second active composition comprising a growth factor, in a suitable carrier or diluent, wherein the combination therapy modulates development and activities of cells and tissues in the subject.

### FIELD OF THE INVENTION

This invention relates to the treatment and prevention of symptoms of diseases and disorders related to impaired development and activities of cells and tissues.

### I. BACKGROUND OF THE INVENTION

Erythropoietin (EPO) is a glycoprotein which serves as the principal factor involved in the regulation of red blood cell synthesis. Erythropoietin is produced in the kidney and acts by stimulating precursor cells in the bone marrow causing them to divide and differentiate into mature red blood cells. The recombinantly produced 165 amino acid glycoprotein has been available for some time as an effective therapeutic agent in the treatment of various forms of anemia, including anemias associated with chronic renal failure, zidovidine treated HIV infected patients, and cancer patients on chemotherapy.

EPO is no longer believed to have exclusive biological activity in the hematopoietic system, and it is now considered to have applicability in a variety of nervous system disorders that can overlap with vascular disease, metabolic impairments, and immune system dysfunction. The receptor for EPO is expressed in the central nervous system. As a result, EPO may offer efficacy for a broad number of disorders that involve Alzheimer's disease, cardiac insufficiency, stroke, trauma, and diabetic complications.

EPO has been explored for the past 7 years for its potential as a neurotherapeutic agent for a wide variety of central nervous system disorders including, but not limited to: multiple sclerosis, amyotrophic lateral sclerosis (ALS) and other motor neuron diseases, seizure disorders, traumatic brain injury, hypoxia, cerebral palsy, spinal cord injury, schizophrenia, pervasive developmental delay, stroke, Parkinson's and Alzheimer's disease autoimmune encephalomyelitis, and other neurological involvement disorders. EPO is considered to have applicability in a variety of nervous system disorders that can overlap with vascular disease, metabolic impairments, and immune system function. Thus, EPO may offer efficacy for a broad number of disorders that involve neurodegenerative disease, cardiac insufficiency, stroke, trauma, and diabetic complications.

A recent report in the literature suggests that EPO may be a potent neuroprotective therapeutic agent for the treatment of ocular diseases that are characterized by retinal ganglion cell (RGC) death. See, Yamasaki M et al., Brain Res.: 1050(1-2):15-26 (2005). Intracerebral and systemic administration of epoetin alfa, the recombinant form of erythropoietin, has been demonstrated to elicit marked neuroprotective effects in multiple preclinical models of CNS disorders. Epoetin alfa has also been shown to prevent the loss of autoregulation of cerebral blood flow in a model of subarachnoid hemorrhage.

During a number of clinical studies, it has been shown that EPO is robust and can prevent metabolic compromise, neuronal and vascular degeneration, and inflammatory cell activation. See, Kenneth Maiese et al, Progress in Neurobiology: 85, 194-213 (2008).

Recent work has elucidated a number of novel cellular pathways governed by EPO that can open new avenues to avert deleterious effects of this agent and offer previously unrecognized perspectives for therapeutic strategies. Obtaining greater insight into the role of EPO in the nervous system and elucidating its unique cellular pathways may provide greater cellular viability not only in the nervous system but also throughout the body.

The use of EPO is not without its considerations especially in light of frequent concerns that may compromise clinical care. One such concern is the many side effects of EPO especially related to the administration of higher dosages of this compound that were shown to be effective to bring about the intended result. These side effects include serious cardio- and thrombovascular events; these events included myocardial infarction, stroke, congestive heart failure, and hemodialysis vascular access thrombosis.

Dosage of erythropoietin is cited to be quite high in research studies to date. In one study, low and high dosage of EPO was administered to subjects with multiple sclerosis resulting in a positive response for the subjects receiving the high dose (48,000 Units weekly) and no response in those receiving the low dose (8000 Units weekly). About 1% of EPO is reported to pass the blood brain barrier when administered by intravenous infusion. See, Hannelore Ehrenreich et. al. Brain 130(10):2577-2588 (2007).

U.S. Patent Application No. 20060058267 discloses methods of treating neurodegenerative diseases and inhibiting neurological damage by administering to a patient in need of such treatment an erythropoietin compound in combination with an N—NOS inhibitor or a GABA-A receptor modulator. This application discloses a synergistic effect of these compounds with EPO while reducing some of the pathological consequences and side effects of any of the compounds administered alone.

While there are clinical benefits in therapies that require administration of EPO in high doses for certain indications, there are nevertheless tremendous risks and pathological side effects associated with these therapies. Additionally, there is a long felt need for combination therapies that synergistically increase and improve the clinical effectiveness of EPO. The invention as disclosed and described herein, overcomes the prior art problems through the generation and use of novel compositions and therapies that synergistically increase effectiveness of EPO while reducing or abolishing the side effects associated with this drug.

### II. SUMMARY OF THE INVENTION

In one aspect, the invention provides methods of preventing, ameliorating, and/or treating symptoms of diseases related to impairment of development and activities of cells and tissues in a subject in need of treatment thereof.

The prevention, treatment and/or amelioration of one or more symptoms of the diseases related to impairment of development and activities of cells and tissues need not be complete, so long as at least one symptom of the disease is prevented, treated or ameliorated. The symptoms of the disease are associated with early, intermediate and/or advance stages of specific diseases, described as infra.

The invention also provides methods of treating a subject at risk for developing symptoms of diseases related to impairment of development and activities of cells and tissues in order to delay the onset of one or more of the underlying etiologies related to this condition.

The inventive methods include administration of a combination therapy comprising at least two active compositions, the first active composition comprising the first active composition comprising a balanced PC and an essential fatty acid composition and the second active composition comprising a growth factor, in a suitable carrier or diluent, wherein the combination therapy modulates development and activities of cells and tissues in the subject.

In one embodiment, the growth factor comprises EPO.

The methods of the invention encompass modulation of activity and development of a wide variety of tissues and cells in the body. The tissues include the general category of epithelial tissues, connective tissues (blood, bone, cartilage), muscle tissues, nerve tissues, stem tissues and cells, and subclasses and categories of these tissues and cells.

A wide spectrum of diseases and disorders are caused or result from the impairment of development and activities of cells and tissues. The diseases and disorders include, by way of example and not limitation, diseases related to central nervous system (CNS), neurological diseases, cardiovascular diseases, stroke, vascular trauma, spinal cord diseases and stenosis, including cervical spinal stenosis and lumbar stenosis, brain diseases, back pains, herniated discs, diseases related to digestive tract, diseases resulting from hyperactivity or hyperplasia of somatic cells, diseases that are mediated by immune system effector cells including cancer and AIDS, diseases related to angiogenesis, inflammatory diseases, eye diseases and disorders related to imbalance and dysfunction of blood cells and tissues, macular degeneration, pervasive developmental delay, seizure disorders, epilepsy, cerebral palsy, brain injury with or without oxygen deprivation, ALS, Autism, Parkinson's Disease, multiple sclerosis, Alzheimer's Disease, Huntington's Disease, respiratory disease, hepatic disease (e.g., hepatic encephalopathy), kidney disease, skin disorders such as gross eczema, Hepatitis C, Lyme disease, Fibromyalgia, chronic fatigue syndrome, post traumatic stress disorder (PTSD), meningitis, encephalitis, and autoimmune diseases, among others.

In one embodiment, the method of the invention is used for prevention, treatment, and/or amelioration of one or more symptoms of a neurodegenerative disease. In another embodiment, the neurodegenerative disease comprises spinal stenosis.

The methods of the invention use erythropoietin as an active composition. The erythropoietin of the invention is in a natural and/or recombinant form. In one embodiment form, the erythropoietin comprises recombinant epoetin alfa.

In one embodiment, the method of the invention uses EPO and PC compositions that are administered contemporaneously or are administered at different time intervals.

In a preferred embodiment, PC is administered both prior to and after EPO administration.

In one embodiment, the PC and EPO compositions are formulated in one or different solutions.

In another embodiment, the first active composition (EPO), the second active composition (PC), or both are administered in a time-released manner.

In one embodiment, the first active composition, the second active composition, or both are in a dry formulation, liquid formulation, or both.

In yet another embodiment, the first active composition is administered at a dosage of about 10,000 to 20,000 units at a weekly or biweekly interval.

In one embodiment, the first active composition, the second active composition, or both are administered parenteraly.

In another embodiment, the first active composition, the second active composition, or both are administered intravenously.

In yet another embodiment, the compositions of the inventions are administered by one intravenous infusion weekly or bimonthly in orderly steps of: i) intravenous administration of about 1500 mg of a balanced PC ii) intravenous administration of about 10,000 units of recombinant epoetin alfa; iii) intravenous administration of about 1500 mg of a balanced PC, wherein the modulation of development and activities of cells and tissues in the subject is commenced after at least one, two, three, four, five, six, seven, or more intravenous infusions.

In another embodiment i) intravenous administration of about 2 to 10 grams of sodium phenylbutyrate; ii) intravenous administration of about 1500 mg of a balanced PC, iii) intravenous administration of about 10,000 units of recombinant epoetin alfa mixed with or without 250 mg of PC; iv) intravenous administration of about 1500 mg of PC; v) intravenous administration of about 10 mg of leucovorin (folinic acid); vi) intravenous administration of about 2000 to 4000 mg of Glutathione, wherein the modulation of development and activities of cells and tissues in the subject is commenced after at least one, two, three, four, five, six, seven, or more intravenous infusions.

In another embodiment the administration of PC and EPO can be in any order as long as the EPO is sandwiched in between the two infusions of PC or at the EPO is mixed with the PC.

In another aspect, the invention provides a composition for prevention, amelioration and/or treatment of symptoms of diseases related to impairment of development and activities of cells and tissues, wherein the composition comprises at least two active ingredients, the first active ingredient comprising erythropoietin and the second active ingredient comprising a balanced PC, in a suitable carrier or diluent.

In one embodiment, the composition of the present invention further includes additional constituents of the PK Protocol, described infra, comprising, linoleic acid and alpha linolenic acid in a ratio of about 4:1, trace minerals, butyrate or phenylbutyrate, electrolytes, methylating agents, glutathione, or a combination thereof.

In yet another aspect, the invention provides a kit for modulation of development and activities of cells and tissues in a subject, comprising: a) a first active composition comprising one or more erythropoietin formulations; b) a second active composition comprising a balanced PC; and c) optionally containing one or more additional constituents comprising: i) linoleic acid and alpha linolenic acid in a ratio of about 4:1; ii) trace minerals; iii) butyrate or phenylbutyrate; iv) electrolytes; v) methylating agents; vi) glutathione; d) instructions for the use of the first and second active compositions and the constituents; and e) instructions for where to obtain any missing components of the kit.

These and other aspects, features and advantages of the present invention will become apparent after a review of the following detailed description of the disclosed embodiments and the appended claims.

### III. DETAILED DESCRIPTION OF THE INVENTION

### DEFINITIONS

As used herein, “modulation” includes, but is not limited to, stabilize, promote, inhibit or disrupt development, differentiation, maturation, proliferation survival, and morphology of target cells and tissues that are implicated in certain disease or disorders subject to the combination therapy of the invention. Modulation also includes interactions between cell types, protein-ligand interactions, e.g., in receptor-ligand recognition. Additionally, modulation includes modulation of activities of cell functions and enzymes including but not limited to: (1) the ability to modulate cell surface recognition; (2) the ability to transduce an extracellular signal (e.g., by interacting with a ligand and/or a cell-surface receptor); (3) the ability to modulate a signal transduction pathway; and (4) the ability to modulate intracellular signaling cascades (e.g., signal transduction cascades). Other modulation of activities may include, e.g., the ability to modulate cellular proliferation; cellular differentiation; chemotaxis and/or migration; and the ability to modulate cell death.

As used herein. “PK Protocol” or “Kane Composition” refers to a specific therapeutic regimen that uses: 1) a phosphatidylcholine formulation, preferably a balanced phosphatidylcholine formulation, including by way of example and not limitation, LipoStabil N™, Essentiale N™, or BodyBio PC; and 2) a balanced ω6 and ω3 fatty acids (i.e. EFA 4:1), and further in combination with one or more of the following agents: 3) rGlutathione; 4) a methylating agent such as for example, Leucovorin, folinic acid, riboflavin, pyridoxine, tetrahydrobiopterin, and/or methylcobalamin; 5) butyrate or sodium phenyl butyrate; 6) electrolytes; 7) minerals; 8) evening primrose oil, 9) Eicosapentaenoic (EPA) rich fish oil. These agents can be administered by a wide variety of routes. PK protocol is administered by a wide variety of ways and modes of administration. In one embodiment, PK Protocol agents are administered by parenteral routes, oral routes or both.

As used herein “balanced PC” refers to specific phosphatidylcholine formulations that contain a more favorable ratio of essential fatty acids (i.e., about 4:1 ω6 and ω3 fatty acids), a higher concentration of phosphatidylcholine, and/or a lower concentration of triglycerides as compared to other PC formulations in the market. Balanced PC includes, by way of example and not limitation, LipoStabil N™, Essentiale N™, and/or BodyBio PC (including both intermediate and end products of BodyBio PC) as described in Example 1.

As used herein, “prevention” includes prevention of one or more symptoms of the diseases disclosed herein. Prevention also includes delaying the onset of one or more symptoms of the diseases disclosed herein. The symptoms of the diseases prevented are associated with early, intermediate and/or advance stages of the disease. The “prevention” provided need not be absolute, i.e., the disease symptoms need not be totally prevented, provided that there is a statistically significant delaying of disease symptoms that have been demonstrated relative to that of a control population.

As used herein, “Glutathione”, and “rGlutathione” (Reduced Glutathione) are used interchangeably herein.

As used herein, an “effective amount” of a composition is an amount sufficient to achieve a desired biological effect, in this case at least one of modulation of activity and/or development of cell populations and/or tissues that are targeted by the combination therapy of the invention. It is understood that the effective dosage will be dependent upon the age, sex, health, and weight of the recipient, the kind of concurrent treatment, if any, frequency of treatment, and the nature of the effect desired. The most preferred dosage will be tailored to the individual subject, as is understood and determinable by one skilled in the art, without undue experimentation.

As used herein, a “subject” is any mammal, in particular a primate, preferably a human, that 1) exhibits at least one symptom associated with impairment of tissue development and activity, or 2) and has been diagnosed with or is at the risk of developing a disease or disorder that causes an impairment of tissue development and activity.

As used herein, a “carrier” refers to a diluent, adjuvant, excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Sterile water is a preferred carrier when the compositions of the invention are administered intravenously. Only aqueous dextrose (5% Dextrose in water) and glycerol solutions, not saline, can be employed as liquid carriers for lipid infusions, particularly for solutions to be infused.

As used herein, the term “about” encompasses values that are within the range of 10% above and 10% below the recited values. For example about 1500 mg includes values of 2000 mg to 1000 mg, and all integer amounts or fractions thereof. The values contemplated within the term “about” do not include Zero.

The invention as described herein provides compositions and methods for preventing, ameliorating and/or treating of diseases and disorders related to impairment of tissue and cell development and activity.

According to one embodiment of the invention, there is provided a combination therapy that prevents, ameliorates and/or treats symptoms of diseases and disorders related to developmental imbalance and dysfunction of cells and tissues, as applied to a wide spectrum of diseases and disorders, listed infra, through application of the PK Protocol in combination with a secondary active agent including, by way of example and not limitation, growth factors, hormones and cytokines.

In one embodiment, the PK protocol has been combined with one or more growth factors, including by way of example and not limitation, Erythropoietin (EPO), Vascular Endothelial Growth Factor (VEGF), Granulocyte Colony Stimulating Factor (G-CSF), Granulocyte Macrophage Colony Stimulating Factor (GM-CSF), Brain Derived Neurotrophic Factor (BDNF), Epidermal Growth Factor (EGF), Sonic Hedgehog (Shh), Fibroblast Growth Factors (e.g., FGF2, FGF8), or a combination thereof.

In one embodiment, the PK protocol or Kane composition is combined with EPO. The novel combination of Kane composition with EPO, which are synergistic when used together, results in easy delivery of and passage of EPO, at much lower concentrations than previously administered, through the cell membrane and the blood brain barrier to support tissue regeneration, to inhibit the inflammatory reactions and to promote cerebral reperfusion.

PK protocol uses 1) a phosphatidylcholine formulation, preferably a balanced phosphatidylcholine formulation, including by way of example and not limitation, LipoStabil N™, Essentiale N™, or BodyBio PC; and 2) a balanced ω6 and ω3 fatty acids (i.e. EFA 4:1), and further in combination with one or more of the following agents: 3) rGlutathione; 4) a methylating agent such as for example, Leucovorin, folinic acid, riboflavin, pyridoxine, tetrahydrobiopterin, and/or methylcobalamin; 5) butyrate or sodium phenyl butyrate; 6) electrolytes; and 7) minerals.

The invention encompasses the use of a balanced PC formulation as an active ingredient in combination with EPO.

PK Protocol has been shown to have significant therapeutic values in preventing, treating or ameliorating the symptoms of diseases and disorders that are related to accumulation of aberrant lipids and toxins that are caused by an imbalance of essential fatty acids. The PK Protocol provides IV and oral treatment protocols that address clearance of possible neurotoxins, yield stabilization of membrane phospholipids and balance the essential fatty acids. The treatment methods of the invention address cellular derangement, and by introducing PC, orally or by IV infusion, potentially offset the accumulation of ceramides, influence fluidity, clear neurotoxins, and stabilize the integrity of the lipid membrane leaflets.

EPO has been shown to be effective for modulation of tissue regeneration and development. There are, however, many side effects associated with EPO and especially to the higher dosages of EPO that were shown to be effective for modulation of tissue regeneration. Only about 1% of EPO is reported to pass the blood brain barrier when administered alone intravenously. The combination therapy of the present invention has unexpectedly completely circumvented the known deleterious side effects associated with the high dose administration of EPO by co-administration of PC and EPO, which in turn increases the bioavailability of this drug and its adsorption and passage through cell membranes and blood brain barrier. Without being limited to a specific mechanism of action, one possible mechanism of action of the combination therapy of the invention is the significant reduction of the blood lipids triglyceride, LDL and cholesterol through the use of PK Protocol, which subsequently thins the blood and facilitates the transport of EPO through cell membranes and the blood brain barrier. Because of the ease and efficiency of transport of EPO that is caused by administration of the PK Protocol, the effective concentration of EPO can be reduced by as much as about 10 to about 100 fold or more without reducing effectiveness of this drug. Reduced dosing of EPO has tremendous clinical advantages in preventing cardio and/or thromboembolic events that are known side effects of any EPO therapy.

By reducing the pathological consequences of erythropoietin when administered in combination with phosphatidylcholine, the overall benefit of the therapeutic intervention is increased considerably. Furthermore, inhibiting multiple pathological processes provided an unexpected synergistic benefit over and above that which was originally achievable with the use of erythropoietin or PC alone.

In one embodiment, the invention, as described and disclosed herein, uses recombinant erythropoietin in combination with a balanced PC as an active composition.

In another embodiment, phosphatidylcholine is administered through one or more different or the same routes of administration in a single or multiple regimen. In a preferred embodiment, phosphatidylcholine is administered twice daily through, IV routes, oral routes, or both. Human trials were conducted on the use of IV bolus phosphatidylcholine to establish the safety of doses of 7 grams, 14 grams and 21 grams in which no side effects were observed. The use of oral and IV phosphatidylcholine facilitates stabilization of phospholipids in cellular membranes thereby addressing hepatic and CNS clearance of neurotoxins.

A dramatic and sustained clinical improvement has been observed within the first few weeks after initiation of oral and intravenous treatment of the PK protocol in combination with EPO in the patient population with one or more symptoms of diseases related to impairment in the development of cells and tissues.

In another embodiment, the invention provides a method of treating or reversing prevalent symptoms of a neurodegenerative disease such as spinal lumbar stenosis by administration of EPO in combination with a balanced PC. The patient receives, for example, at least one or two infusions daily for about 1, 2, 3, 4, 5, 6, or 7 weeks or more. The inventive bolus dosing with the balanced PC as an intravenous push or long drip followed by an infusion of EPO and a second infusion of the balanced PC has yielded significant results in support of the regeneration of spinal cord tissues, and marked neurological improvement in patients with Multiple Sclerosis, Alzheimer's, Autism, Dementia, Post Stroke, Parkinson's and ALS. The concept of the IV use of PC is that of rejuvenating membranes and cells and an attempt to promote a consummate increase in fluidity due to the high concentration of essential fatty acids with a multitude of cis-double bonds within the PC.

In one embodiment, the combination therapy of the invention, having a low dose EPO and PC, has been administered once weekly to patients demonstrating one or more symptoms of a CNS-related disease with marked improvement in patient's presentation and complete recovery from one or more symptoms after 3-12 weeks.

Brain and spinal cord injury caused by neurodegenerative diseases often result in lifelong disability and premature death. The cause of disability and death is the disruption of function and frank death of neurons and other cells in the central nervous system. Therefore, a clear benefit is anticipated from therapies that reduce or prevent neuronal dysfunction and death after ischemic, hypoxic or traumatic CNS insult.

One of the causes of neuronal dysfunction and death after CNS insult is toxicity caused by a prolonged elevation of glutamate and other excitatory amino acids (EAAs) and overactivation of the N-methyl-D-aspartate (NMDA) subtype of glutamate receptors. Glutamate and other EAAs play dual roles in the central nervous system as essential amino acids and the principal excitatory neurotransmitters. There are at least four classes of EAA receptors, specifically N MDA, AM PA (2-amino-3-(methyl-3-hydroxyisoxazol-4-yl) propanoic acid), kainate and metabotropic. These EAA receptors mediate a wide range of signaling events that impact all physiological brain functions. As neurotransmitters, EAAs are released from postsynaptic nerve terminals and then are rapidly resequestered by a variety of cellular reuptake mechanisms. Consequently, the physiological levels of EAAs in the brain parenchyma are maintained at a low level. However, after a CNS insult, the levels of EAAs in the parenchyma increase dramatically and may remain elevated for periods of hours to days. This results in pathological overactivation of EAA receptors and neuronal dysfunction and death.

In one embodiment, the present invention provides methods and compositions for prevention, amelioration and/or treatment of one or more symptoms of spinal neurodegenerative diseases, specifically cervical and lumbar spinal stenosis. Without being limited to any specific mechanism of action, one possible mechanism of action of the combination therapy is by inhibiting the inflammatory reaction and promoting cerebral reperfusion around the site of injury in spinal cord.

In another embodiment, the present invention is directed to inflammation-associated tissue damage and is particularly directed to therapeutic methods for treating localized and systemic inflammation by mobilizing, enhancing the trafficking of, and/or recruiting stem cells and/or progenitor cells to the wound or damaged tissue site, as well as the treatment of a variety of diseases associated with the inflammation, such as a wound or cancer.

The combination therapy of the invention is also beneficial for cancer patients who have been through chemotherapy and/or radiotherapy. In one embodiment, the combination therapy is used to treat anemia due to concomitant myelosuppressive chemotherapy. In another embodiment, the combination therapy of the invention is directed to therapeutic methods involving intravenous introduction of EPO and PC to certain target cell populations, such as smooth muscle cells, cancer cells, and/or somatic cells requiring modulation of tissue development to ameliorate a disease state and effector cells of the immune system. The combination therapy of the invention is also particularly beneficial for treating conditions such as stenosis following vascular trauma or disease, diseases resulting from hyperactivity or hyperplasia of somatic cells and diseases that are mediated by immune system effector cells including cancer and AIDS. Intravenous introduction of the active compositions of the invention capable of modulating proliferation, migration, or contraction of smooth muscle is also described. The invention also relates to the direct or targeted delivery of the active compositions to vascular smooth muscle cells that result in dilation and fixation of the vascular lumen (biological stenting effect).

For each of the above-recited methods of the present invention, the therapeutically effective amount of a balanced PC, with or without additional agents of PK Protocol, in combination of EPO and a pharmaceutically acceptable excipient thereof may be administered to a subject in need thereof in conjunction with a therapeutically effective amount of one or more anti-viral drugs, antibacterial drugs and/or anti-inflammatory compounds and/or a therapeutically effective amount of one or more immunomodulatory agents.

In certain embodiments of the methods of the present invention, the anti-inflammatory compound or immunomodulatory drug comprises interferon; interferon derivatives comprising betaseron, beta.-interferon; prostane derivatives comprising iloprost, cicaprost; glucocorticoids comprising cortisol, prednisolone, methyl-prednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; intravenous gamma globulin (IVIG), soluble TNF-receptors; TNF-antibodies; soluble receptors of interleukins, other cytokines, T-cell-proteins; antibodies against receptors of interleukins, other cytokines, T-cell-proteins; and calcipotriols and analogues thereof taken either alone or in any combination thereof.

In yet another aspect, the present invention is directed to a method of relieving or ameliorating cancer or tumor development, metastasis, protein degradation, cell proliferation in cancer cells, and/or inhibition of symptoms associated with cancer diseases and/or cancer indications in a mammal suffering from cancer diseases or cancer indications, which method comprises administering to the mammal in need thereof a therapeutically effective amount of the combined EPO and a balanced PC, optionally containing one or more additional agents of PK protocol, and a pharmaceutically acceptable excipient, either alone or in combination with one or more anti-inflammatory compounds or immunomodulatory agents, wherein said compositions exhibiting modulation of tissues regeneration activities and detoxification when applied three days or more after administration of chemotherapy or radiation to clear the toxicity of these interventions, and are sufficient to inhibit the cancer or tumor development, metastasis, protein degradation, and/or cell proliferation in cancer cells.

The modulation of development and activity of cells and tissues for all methods described herein is on the order of about 15-100%. In one embodiment, the modulation of development and activity of cells and tissues is on the order of about 20-30%. In yet another embodiment, the modulation of development and activity of cells and tissues is on the order of about 40-50%. In another embodiment, the modulation of development and activity of cells and tissues is on the order of about 60-70%. In another embodiment, the modulation of development and activity of cells and tissues is on the order of about 80-90%. In another embodiment, the modulation of development and activity of cells and tissues is on the order of about 90-100%. It is intended herein that the ranges recited also include all those specific percentage amounts between the recited ranges. For example, the range of about 90 to 100% also encompasses 96 to 99%, 91 to 95%, etc, without actually reciting each specific range therewith.

In one embodiment, the present invention provides methods for preventing, treating and/or ameliorating one or more symptoms of neovascular diseases of the eye, including for example, neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization. The method comprises administering EPO and a balanced PC in a therapeutically-effective amount to prevent, ameliorate and/or treat eye and central nervous system diseases or injuries, such as age-related macular degeneration and other central nervous system degenerative diseases, photic injury, ischemic diseases, and inflammatory diseases.

In another embodiment, the invention provides methods for preventing, treating and/or ameliorating one or more symptoms of primary central nervous system (CNS) lymphoma, which is a rare cancer. This cancer involves the central nervous system (brain, spinal cord, one or both eyes, and/or the coverings of the brain and optic nerve, also known as the meninges). The designation as a “lymphoma” reflects the fact that the cancerous cells are lymphocytes, a type of white blood cell. Primary CNS lymphoma affects all age groups, but is most commonly diagnosed in persons who are over 50 years of age. In addition, individuals who are immunosuppressed such as patients with AIDS or those taking certain drugs after organ transplantation, appear to be at increased risk.

### 1. Erythropoietin

Erythropoietin (EPO) is a glycoprotein which stimulates red blood cell production. It is produced in the kidney and stimulates the division and differentiation of committed erythroid progenitors in the bone marrow. The glycoprotein is administered parenterally, for example, as an intravenous (IV) or subcutaneous (SC) injection. In one embodiment, the parenteral formulations in use are conventional sterile buffered aqueous solutions for IV or SC injection which contain human serum albumin (HSA) as a carrier. Such formulations are marketed in the United States under the trade names EPOGEN™ and PROCRIT®. These products contain erythropoietin in 1 ml single dose, preservative-free or 2 ml multidose preserved vials.

The combination therapy of the present invention encompasses the use of erythropoietin in both its natural and recombinant forms. One recombinant form of EPO is PROCRIT® (epoetin alfa) that is a 165 amino acid glycoprotein manufactured by recombinant DNA technology and has the same biological effects as endogenous erythropoietin. It has a molecular weight of 30,400 daltons and is produced by mammalian cells into which the human erythropoietin gene has been introduced. The product contains the identical amino acid sequence of isolated natural erythropoietin. PROCRIT® is formulated as a sterile, colorless liquid in an isotonic sodium chloride/sodium citrate buffered solution or a sodium chloride/sodium phosphate buffered solution for intravenous (IV) or subcutaneous (SC) administration. Epoetin treatment offers an attractive but costly alternative to red blood cell transfusion for managing anemia associated with several diseases including cancer.

The effective dose of EPO when administered alone is reported to be 150-200 Units/kg SC three times a week (TIW) or up to 100,000 Units SC weekly.

Recombinant human erythropoietin (epoetin alfa) has proven beneficial for the treatment of various anemias. The mechanism of action of endogenous erythropoietin and the therapeutic use of epoetin alfa to stimulate red blood cell production and improve the quality of life in cancer patients are reviewed here. Epoetin alfa may also attenuate the cognitive dysfunction associated with cancer therapy. Interestingly, functional endogenous erythropoietin receptor signaling pathways have been demonstrated in numerous nonerythropoietic tissues. Of particular importance, epoetin alfa confers neurotrophic and neuroprotective effects in cultured neurons and in several animal models for neurologic disease. In one clinical trial, epoetin alfa appeared to limit functional and histologic damage in patients with stroke. Therefore, in cancer patients receiving chemotherapy, the beneficial effects of epoetin alfa could be mediated not only through enhanced erythrocyte production but also via direct effects on the nervous system. Further investigation into the non-erythropoietic effects of epoetin alfa could broaden its clinical utility for patients with cancer and also provide new therapies for various neurologic disorders.

Anaemia, often associated with chemotherapy, is a common and debilitating disorder in cancer patients. Recombinant human erythropoietin (epoetin alfa) was introduced in the 1990s for the treatment of chemotherapy-related anaemia. Data from randomised, double-blind, placebo-controlled studies and large, non-randomised, community-based studies have demonstrated that either of the FDA-approved dosing schedules of epoetin alfa 150-300 U/kg three times weekly or 40,000-60,000 U/week s.c., significantly increases haemoglobin levels, reduces transfusion requirements, and improves quality of life in anaemic cancer patients undergoing chemotherapy or chemoradiation therapy.

EPO has also been reported to possess neuroprotection activities. The mechanisms of EPO-induced neuroprotection include, by way of example and not limitation, prevention of glutamate-induced toxicity, inhibition of apoptosis, anti-inflammatory effects, antioxidant effects, and stimulation of angiogenesis. Collectively, these findings suggest that EPO may have potential therapeutic utility in patients with ischemic CNS injury.

Dosage of erythropoietin is cited to be quite high in research studies to date. In one study low and high dosage of EPO was administered to subjects with multiple sclerosis resulting in a positive response for the subjects receiving the high dose (48000 Units weekly) and no response in those receiving the low dose (8000 Units weekly).

2. Components of PK protocol

2.1 Phosphatidylcholine

Phosphatidylcholine (PC) is the predominant phospholipid of all cell membranes and of the circulating blood lipoproteins. Of the tens of thousands of molecules that make up the life of a cell, Phosphatidylcholine (PC) stands apart; probably the most important one of all. PC is the main lipid constituent of the lipoprotein particles circulating in the blood and the preferred precursor for certain phospholipids and other biologically important molecules. PC also provides antioxidant protection in vivo. In animal and human studies, PC protected against a variety of chemical toxins and pharmaceutical adverse effects.

Chemically, PC is a glycerophospholipid that is built on glycerol (CH2OH-CHOH-CH2OH) and substituted at all three carbons. Carbons I and 2 are substituted by fatty acids and carbon 3 by phosphorylcholine. Simplistically, the PC molecule consists of a head-group (phosphorylcholine), a middle piece (glycerol), and two tails (the fatty acids, which vary). Variations in the fatty acids in the tails account for the great variety of PC molecular species in human tissues.

In vivo, PC is produced via two major pathways. In the predominant pathway, two fatty acids (acyl “tails”) are added to glycerol phosphate (the “middle piece”), to generate phosphatidic acid (PA) that is converted to diacylglycerol, after which phosphocholine (the “head-group”) is added on from CDP-choline. The second, minor pathway is phosphatidylethanolamine (PE) methylation, in which the phospholipid PE has three methyl groups added to its ethanolamine head-group, thereby converting it into PC.

Taken orally, PC is very well absorbed, up to 90% per 24 hrs when taken with meals. PC enters the blood gradually and its levels peak over 8-12 hours. During the digestive process, the position-2 fatty acid becomes detached (de-acylation) in the majority of the PC molecules. The resulting lyso-PC readily enters intestinal lining cells, and is subsequently re-acylated at this position. The position-2 fatty acid contributes to membrane fluidity (along with position 1), but is preferentially available for eicosanoid generation and signal transduction. The omega-6/omega-3 (ω6 or ω3) balance of the PC fatty acids is subject to adjustment via dietary fatty acid intake. Choline is most likely an essential nutrient for humans, and dietary choline is ingested predominantly as PC. Greater than 98 percent of blood and tissue choline is sequestered in PC that serves as a “slow-release” blood choline source.

Methyl group (—CH3) availability is crucial for protein and nucleic acid synthesis and regulation, phase-two hepatic detoxification, and numerous other biochemical processes involving methyl donation. Methyl deficiency induced by restricted choline intake is linked to liver steatosis in humans, and to increased cancer risk in many mammals. PC is an excellent source of methyl groups, supplying up to three per PC molecule, and is the main structural support of cell membranes, the dynamic molecular sheets on which most life processes occur. Comprising 40 percent of total membrane phospholipids, PC's presence is important for homeostatic regulation of membrane fluidity. PC molecules of the outermost cell membrane deliver fatty acids on demand for prostaglandin/eicosanoid cellular messenger functions, and support signal transduction from the cell's exterior to its interior.

A wide variety of PC formulations and compositions can be used within the scope of the invention. Preferred PC compositions used within the scope of the invention include, by way of example and not limitation, compositions comprising a balanced phosphatidylcholine formulation including Essentiale N™, LipoStabil N™, and/or BodyBio PC. In a preferred embodiment, the phosphatidylcholine of the invention is BodyBio PC available from BodyBio Inc. (Millville, N.J. USA). The concentration of PC in IV administration ranges from about 100 mg to about 10,000 mg. In one embodiment, the concentration range of PC is from about 200 mg to about 5000 mg. In another embodiment, the concentration range of PC is from about 300 mg to about 3000 mg. In a preferred embodiment of the invention, the concentration range of PC is from about 500 mg to about 1000 mg.

The total amount of phospholipids in BodyBio PC is 66.8%, which is about 12% higher than competitive PC products (i.e., 66.8% versus 52.5%). The increased level of available phospholipids is a significant improvement over the competitive PC products. Bodybio PC has an additional advantage of containing phosphatidyl ethanolamine (PE) and phosphotidylinositol (PI). These compounds are both very beneficial for health and are present in the natural phosphatidylcholine compounds in the body.

2.2 Essential Fatty Acids (EFAS)

Essential Fatty Acids (EFAs) are long-chain polyunsaturated fatty acids derived from alpha linolenic acid and linoleic acid. EFAs are necessary fats that humans cannot synthesize, and must be obtained through diet. EFAs compete with undesirable fats (e.g. trans fats and cholesterol) for metabolism. Also, EFAs raise the HDL (High Density Lipoprotein) that is also considered beneficial for the body by capturing the undesirable LDL (Low Density Lipoprotein), and escort it to the liver where it is broken down and excreted.

Essential fatty acids used within the scope of the invention include Linoleic acid and alpha linolenic acids D-isomers, L isomers, racemic mixtures, non-racemic mixtures, conjugated linoleic acid (CLA) containing abundant isomers c9, t11, t10, and c12-CLAs, or a combination thereof.

There are two families of EFAs: Omega-3 and Omega-6. Omega-9 is necessary yet “non-essential” because the body can manufacture it in a modest amount, provided essential EFAs are present. The number following “Omega-” represents the position of the first double bond, counting from the terminal methyl group on the molecule. Omega-3 fatty acids are derived from Linolenic Acid, Omega-6 from Linoleic Acid, and Omega-9 from Oleic Acid.

EFAs support the cardiovascular, reproductive, hepatic, immune, and nervous systems. The human body needs EFAs to manufacture and repair cell membranes, enabling the cells to obtain optimum nutrition and expel harmful waste products. A primary function of EFAs is the production of prostaglandins, which regulate body functions such as heart rate, blood pressure, blood clotting, fertility, conception, and play a role in immune function by regulating inflammation and encouraging the body to fight infection. Essential Fatty Acids are also needed for proper growth in children, particularly for neural development and maturation of sensory systems, with male children having higher needs than females. Fetuses and breast-fed infants also require an adequate supply of EFAs through the mother's dietary intake. Because high heat destroys linolenic acid, cooking in linolenic-rich oils or eating cooked linolenic-rich fish is unlikely to provide a sufficient amount.

EFA deficiency is common in the United States, particularly Omega-3 deficiency and now Omega-6 deficiency due to the increased use of hydrogenated vegetable oil, and recently, over prescribing and consumption of fish oil. Essential fatty acid supplements include solutions comprising a mixture of omega 6 and omega 3 fatty acids, in ratio of from about 20:1, 10:1, 5:1, 4:1, 3:1, 2:1, 1:1, or less. It is intended herein that by recitation of such specified ranges, the ranges recited also include all those specific integer amounts between the recited ranges. For example, in the range of about 4:1, it is intended to also encompass 4.2:1, 3.8:1, 3.5:1, 3.2:1, 3:1, etc, without actually reciting each specific range therewith. Preferably the ratio between the omega 6 and omega 3 fatty acids is about 4:1 v/v.

Incorporating the 4:1 ratio requires consideration of the weaker human FA (fatty acids) capability which necessitates the essential addition of dietary HUFA (highly unsaturated fatty acids) support such as meat, dairy, egg yolk, seafood, or fish oil supplements. The principal value of the 4:1 ratio is the ability to raise the level of fluidity with a low risk of over-expression of either ω6 or ω3 FAs. Clinical application of EFA 4:1 gives the clinician a critically important tool to raise EFAs and subsequently fluidity to a higher level and maintain that critical balance. Balancing EFAs with about 80% ω6s will in effect contribute to the formation of Arachidonic acid (AA).

AA (20:4ω6) is a 20 carbon HUFA with 4 double bonds and is the lead eicosanoid for the production of prostaglandins, thromboxanes and leucotrienes. Arachidonic acid (AA) is a prominent essential fatty acid in red blood cells as 15% and total brain lipids are comprised of 12% AA. All fluidity comes from the double bonds (DB) of the MUFA (monounsaturated fatty acids), PUFA (polyunsaturated fatty acids), and HUFA (highly unsaturated fatty acids) with the most prominent coming from the ω6s. A review of the melting point of each lipid helps to visualize the contribution of the DBs. Palmitic and stearic, both saturated have a melting point of about 65° C. and it accounts for about 32% of the red cell membrane. Since the body has a temperature of 37.5° C., palmitic acid (PA) and stearic acid (SA) are solid in the membrane of animals. Oleic acid (OA), a monounsaturated FA with one DB is liquid at 16° C., it accounts for about 10.2% of red cell fatty acids and is the beginning of fluidity.

TABLE 1
EFAs, double bonds, fluidity contribution, melting point.

| | Double | % of Red | Total DBs | Melting |
|---|---|---|---|---|
| | Bonds | Blood Cells | (Dbs × %) | Point |
| The ω6s: | | | | |
| Linoleic (LA) | 2 DB | 10.28% | 20.56 | −5° C. |
| Gamma Linolenic (GLA) | 3 DB | 0.07% | 0.21 | −11° C. |
| Dihomogamma Linolenic | 3 DB | 1.47% | 4.41 | −11° C. |
| Arachidonic (AA) | 4 DB | 15.07% | 60.28 | −49° C. |
| Adrenic | 4 DB | 3.48% | 13.94 | |
| The ω3s: | | | | |
| Alpha Linolenic | 3 DB | 0.28% | 0.9 | −11° C. |
| Eicosapentaenoic | 5 DB | 0.44% | 2.2 | −55° C. |
| Docosapentaenoic | 5 DB | 2.06% | 10.3 | |
| Docosahexaenoic | 6 DB | 3.46% | 20.76 | −59° C. |

Multiplying the DBs times the percent fatty acid concentration; the total value for the ω6s is 99.40 compared to the ω3s at 34.16. Clearly the ω6s are the prominent FA in the human body with close to 3 times the energy value of the ω3s. The numbers reverse themselves with the ω3 s taking prominence in the brain with the much higher concentration of DHA at about 17-22% and especially in the outer segments of the photo receptor cells in the retina at about 55%. Viewing the double bonds as a storehouse of energy presents a different picture of AA than currently held in the popular literature. The disturbing picture of AA is grossly misrepresented as is its metabolic value. Lacking sufficient arguments for any suppression of arachidonic acid as well as the suppression of any other FAs, we have found that the proper balance of the fatty acids must be made on a case by case basis on the basis of the individualized biochemical data, such as for example, individual's red cell lipid analysis. However, the promiscuous use of marine oil, as is the case in a surprising number of patients, has resulted in gross distortion of their red cell fatty acid profiles.

Over the past 10 years, the phenomenon of an omega 3 overdose syndrome has been prevalent. More common symptoms in pediatric patients are hypotonia and lethargy (if high EPA formulas were used), eczema or other skin eruptions, inflammation, lack of speech, poor responsiveness, learning difficulties, irritability, and seizures. Pediatric patients appear to have significant re-stabilization of arachidonic acid, more slowly with GLA and DGLA, with aggressive oral balanced HUFA lipid therapy (egg yolk, meat fat, evening primrose oil) within about 6 months from the time that marine oil has been overdosed.

The phospholipid therapy of the PK Protocol of the invention expedites stabilization of balanced phospholipids in the membrane in both our adult and pediatric populations. In one embodiment, the treatment is via IV administration of a phosphatidylcholine derived from soy composed of 50% dilinoleoylphosphatidylcholine.

The use of excessive quantities of marine or flax oil can suppress the ω6s, reflected in the lower concentration of AA (arachidonic acid) which can disturb the balance of eicosanoids. As research emerges on the complexity of the interaction of the higher order ω6 to ω3, it is becoming more evident that balance of ω6 to ω3 is paramount. It has been found that when EPA was supplemented but not other long-chain n-3 or n-6 PUFA there was a decrease natural killer cell activity in healthy subjects. When arachidonic acid is suppressed due to excess intake of omega 3, toxicity or disease, the body is perturbed which is clearly viewed in the patient's red cell fatty acid analysis. Arachidonic acid is preferentially wasted in states of heavy metal toxicity and has been observed to be sharply suppressed in red cell fatty acid analysis in states of heavy metal toxicity (Kane et al., 2002a). Arachidonic acid is reduced in serum concentrations in pregnant women and their infant's cord blood with exposure to polychlorinated biphenyls (PCBs) indicative of desaturase inhibition.

2.2.1 Omega-3 Fatty Acids Alpha Linolenic Acid (ALA) is the principal Omega-3 fatty acid, which a healthy human will convert into eicosapentaenoic acid (EPA), and later into docosahexaenoic acid (DHA). Omega-3s are used in the formation of cell membranes, making them supple and flexible, and improving circulation and oxygen uptake with proper red blood cell flexibility and function.

Omega-3 deficiencies are linked to decreased memory and mental abilities, tingling sensation of the nerves, poor vision, increased tendency to form blood clots, diminished immune function, increased triglycerides and increased “bad” cholesterol (LDL) levels, impaired membrane function, hypertension, irregular heart beat, learning disorders, menopausal discomfort, and growth retardation in infants, children, and pregnant women.

Food containing alpha linolenic acid includes flaxseed oil, flaxseed, flaxseed meal, hempseed oil, hempseed, walnuts, pumpkin seeds, Brazilian nuts, sesame seeds, avocados, some dark leafy green vegetables (e.g., kale, spinach, mustard greens, collards, etc.), canola oil (cold-pressed and unrefined), soybean oil, and others. Higher order omega 3 fatty acids (HUFA) sources include wild salmon, mackerel, sardines, anchovies, albacore tuna, cod liver oil, fish oil, and other cold water fish. Foods rich in higher order—HUFA omega-3 fatty acids—as wild salmon and sardines are suggested to the subjects as part of their diet.

In one embodiment, four parts of linoleic acid omega-6 oil as cold pressed, organic sunflower oil is utilized with one part of alpha linolenic acid as cold pressed, organic flaxseed oil as a 4:1 omega 6 to omega 3 ratio balanced oil.

2.2.2. Omega-6 (Linoleic Acid)

Linoleic Acid is the primary Omega-6 fatty acid. A healthy human with good nutrition will convert linoleic acid into gammae linolenic acid (GLA), which will later be synthesized into the eicosanoid (diehomogamma linolenic acid) DGLA from the Omega-6 group into prostaglandin onee series.

Eicosanoids are hormone-like compounds, which aid in many bodily functions including vital organ function and intracellular activity. Omega-6 DGLA and Arachidonic acid (AA), along with omega-3 EPA are all eicosanoids that emerge into prostaglandins one, two and three.

Some Omega-6s improve diabetic neuropathy, rheumatoid arthritis, PMS, skin disorders (e.g. psoriasis and eczema), inflammation, allergies, autoimmune conditions and aid in cancer treatment. Food containing linoleic acid includes safflower oil, sunflower seed, sunflower oil, hempseed oil, hempseed, pumpkin seeds, borage oil, evening primrose oil, black currant seed oil, among many others.

2.3. Methylating Agents

Methylating agents donate methyl groups to molecules to enhance or reduce their expression. One important function of methylating agents is in cellular regeneration; growth and repair per stimulation of DNA expression. Another important function of methylating agents is to selectively “rescue” normal cells from the adverse effects of methotrexate or other poisonous substances via detoxification. Other functions of methylating agents involve stabilization of phospholipids on the cell membrane, binding of neurotransmitters to their receptors and impeding the ability of cancer cells to divide.

Encompassed within the scope of the claimed invention are several types and classes of methylating agents. In a preferred embodiment of the invention, the methylating agent is in a natural form or derived from a natural source. Such natural methylating agents include, by way of example and not limitation, agents within the family of vitamin B group of vitamins including Methylcobalamin, Leucovorin/Folinic Acid, Tetrahydrobiopterin, Pyridoxine, Riboflavin or a combination thereof.

Disturbances in methylation pathways may occur after exposure to heavy metals, thimerosal (preservative in vaccinations), large quantities of alcohol, or chemicals or medication (terbutaline). See, for example, in MOLECULAR ORIGINS OF HUMAN ATTENTION—THE DOPAMINE—FOLATE CONNECTION by Richard C. Deth (Kluwer Academic Publishers: Norwell, Mass., (2003)), incorporated herein by reference in its entirety. Dr. Deth, describes damage to the enzyme methionine synthase after exposure to heavy metals and alcohol whereby the enzyme may be stimulated by the use of the methylated B vitamins methylcobalamin tetrahydrofolate, folinic acid, and tetrahydrobiopterin. A direct connection between polymorphism resulted from toxic exposures to the enzyme methylene tetrahydrofolate reductase (MTHFR) has also been widely documented in the literature. If methylation pathways are not supported with methylated forms of the B vitamins folinic acid and methylcoblamin, the ability to detoxify, balance hormones, stabilize cell membrane functions, rejuvenate, DNA expression, and to lock neurotransmitters such as dopamine and serotonin to their receptors is grossly impaired.

2.3.1. Methylcobalamin

Methylcobalamin is a type of Vitamin B12. Vitamin B12 has several different formulations including hydroxy, cyano, and adenosyl, but only the methyl form is used in the central nervous system. Deficiency states are fairly common and vitamin B12 deficiency mimics many other disease states of a neurological or psychological kind, and it causes anemia. B12 is converted by the liver into methylcobalamin but not in therapeutically significant amounts. Vitamin B12 deficiency is caused by a wide range of factors including low gastric acidity (common in older people), use of acid blockers such as Prilosec™ or excessive laxative use, lack of intrinsic factor, poor absorption from the intestines, lack of Calcium, heavy metal toxicity, excessive Vitamin B12 degradation, internal bleeding, excessive menstrual flow, exposure to high amounts of alcohol, or damage to methylation pathways/enzymes such as methylene tetrahydrofolate reductase (MTHFR) due to toxicity exposure, among others.

Methylcobalamin donates methyl groups to the myelin sheath that insulates nerve fibers and regenerates damaged neurons. In a B12 deficiency, toxic fatty acids destroy the myelin sheath but high enough doses of B12 can repair it. Methylcobalamin is better utilized and retained than other forms of B12 (such as cyanocobalamin). Methylcobalamin protects nerve tissue and brain cells and promotes healthy sleep and is a cofactor of methionine synthase, which reduces toxic homocysteine to the essential amino acid methionine. Methylcobalamin also protects eye function against toxicity caused by excess glutamate.

The accumulation of very long chain fatty acids (VLCFAs) and the resulting formation of ceramides in the brain/CNS may reflect impaired detoxification in methylation. To date every child with ASD and PDD tested for MTHFR (methylene tetrahydrofolate reductase) mutation has had a positive result for C677T, A1298C or both. The phenomenon of disturbed peroxisomal function is not limited to autism and PDD, but has been observed in our patients with ALS, MS, Parkinson's Disease, Post Stroke, AIDS, Alzheimer's, seizure disorders and toxicity states after exposure to neurotoxic environmental mold, heavy metals, methylmercury in fish, pesticides, chemicals and microbial infections.

There are striking relationships of toxic exposure (chemicals, heavy metals) and autism to disruption in methylation pathways. Impaired methylation capacity in children with autism implicates metabolic imbalance. Disturbances in methylation can result in impaired detoxification, altered genetic expression, suppressed growth and repair, poor binding of dopamine and serotonin to their receptors, which require a methyl group in their headgroup of their phospholipid for a stable connection to the cell membrane.

2.3.2. Leucovorin, Tetrahydrobiopterin, Folinic Acid

Leucovorin is the active form of the B complex vitamin, Folinic acid. Leucovorin is used as an antidote to drugs that decrease levels of Folinic Acid. Folinic Acid assists the formation of red and white blood cell and the synthesis of hemoglobin. Some treatments require what is called leucovorin rescue, because the drug used to treat the cancer or other infection has had an adverse effect on Folinic Acid levels. Leucovorin is used to reduce anemia in people taking dapsone. Leucovorin is also taken to decrease the bone marrow toxicity of sulfa drugs, and in combination with pyrimethamine to decrease the toxicity of toxoplasmosis treatment. Leucovorin is also used in combination with trimetrexate to prevent bone marrow toxicity and in combination with chemotherapeutic agents such as methotrexate. Other substituents for Leucovorin include Citrovorum, Wellcovorin, and/or folinic acid, among others.

Leucovorin calcium (Folinic acid) is a reduced form of folic acid. It is usually used 24 hours after methotrexate to selectively “rescue” normal cells from the adverse effects of methotrexate caused by inhibition of production of reduced folates. It is not used simultaneously with methotrexate, as it might then nullify the therapeutic effect of the methotrexate. More recently, leucovorin has also been used to enhance the activity of fluorouracil by stabilizing the bond of the active metabolite (5-FdUMP) to the enzyme thymidylate synthetase. Commercially available Leucovorin is the racemic mixture of D and L isomers. It is now recognized that the activity of Leucovorin is due to the L form. The invention disclosed herein encompasses the use of both L and or D isomers and any racemic mixtures thereof.

In one embodiment, the treatment method of the invention comprises administration of oral folinic acid (e.g., about 5 to 10 mg.) and methylcobalamin (e.g. about 2 to 5 mg.) in patients with autistic spectrum disorder. Increased dosage resulted in more positive outcomes, especially along with methylcoblamin intramuscularly, Leucovorin (folinic acid), or a combination thereof. In a preferred embodiment, Leucovorin is administered by IV infusion and methylcoblamin is administered intramuscularly. By supporting methylation via methylcobalmin and folinic acid, the treatment methods of the invention amplify detoxification as well as stabilizing membrane function.

2.3.3. Synthetic Methylating Agents

Synthetic methylating agents, which impair the ability of malignant cells to divide, include dacarbazine (DTIC), temozolomide (TMZ), procarbazine, Methylnitrosourea, N-methyl-N-nitrosourea (MNU), methyl methanesulfonate (MMS) and methyl iodide, among others.

2.4 Glutathione

Reduced Glutathione (rGlutathione) is known chemically as N—(N-L-gamma-glutamyl-L-cysteinyl) glycine and is abbreviated as GSH. Its molecular formula is C10OH17N3O6S and its molecular weight is 307.33 Daltons. Glutathione disulfide is also known as L-gamma-glutamyl-L-cysteinyl-glycine disulfide and is abbreviated as GSSG. Its molecular formula is C20H32N6O12S2. The term glutathione is typically used as a collective term to refer to the tripeptide L-gamma-glutamyl-L-cysteinylglycine in both its reduced and dimeric forms. Monomeric glutathione is also known as reduced glutathione and its dimer is also known as oxidized glutathione, glutathione disulfide and diglutathione. Reduced glutathione is also called glutathione and the glutathione dimer is referred to as glutathione disulfide.

Glutathione is widely found in all forms of life and plays an essential role in the health of organisms, particularly aerobic organisms. In animals, including humans, and in plants, glutathione is the predominant non-protein thiol and functions as a redox buffer, keeping with its own SH groups proteins in a reduced condition, among other antioxidant activities.

Glutathione plays roles in catalysis, metabolism, signal transduction, gene expression and apoptosis. It is a cofactor for glutathione S-transferases, enzymes which are involved in the detoxification of xenobiotics, including carcinogenic genotoxicants, and for the glutathione peroxidases, crucial selenium-containing antioxidant enzymes. It is also involved in the regeneration of ascorbate from its oxidized form, dehydroascorbate.

Glutathione functions as an antitoxin as well as antioxidant and is extremely important for the protection of major organs, the function of the immune system, and the fight against aging. It minimizes the damage caused by free radicals that is important for the health of cells. Recent, extensive research has shown the direct relationship between decreased glutathione levels and the progression of many chronic diseases. It is reported that decreased Glutathione may be a result of various types of prolonged stress and hyperactivity of the immune system, which in turn compromises the health of the body's cells. Unfortunately, taking Glutathione (L-Glutathione capsules) orally is not a suitable method for replacement of losses since the glutathione molecule is very unstable and is destroyed by the stomach acid before it can be absorbed.

Glutathione's major effect is intracellular, and intra-organelle. Within the mitochondria Glutathione is present in tissues in concentrations as high as one millimolar. There are undoubtedly roles of glutathione that are still to be discovered.

2.5 Sodium Phenylbutyrate (PBA)

Butyrate is an important short chain fatty acid that provides fuel for colon cells and may help protect against colon cancer. The most potent dietary source of butyrate is reported to be butter (3%). Butyrate is made in the colon by bacteria. Antibiotics kill the bacteria that produce butyrate. Butyrate has a particularly important role in the colon, where it is the preferred substrate for energy generation by colonic cells.

Butyrate has been shown to significantly inhibit the growth of cancerous colon cells. Scientists have found a human gene that stops the growth of cancer cells when activated by fiber processing in the colon. Whether by supplement or by enema, a few pilot studies suggest that the presence of butyrate in colon is useful in reducing symptoms and restoring indicators of colon health in ulcerative colitis, but one study showed no benefit over placebo. Several doctors claim that many people are helped with butyrate enemas. Butyrate levels are commonly measured in comprehensive stool analyses and act as a marker for levels of beneficial bacteria.

One possible mechanism of action of butyrate is through breaking up ceramides which accumulate in the membrane as clusters called “lipid rafts”. Rafts are composed of ceramides, cholesterol and sphingomyelin (SM) all of low energy with either very long chains or rigid chains (e.g. cholesterol.) Ceramides are generally structured with lipid tails as very long chain fatty acids (VLCFAs) and combine with PC to form SM (reversible back into ceramide and phosphatidylcholine). SM maintains the VLCFAs from the ceramide as opposed to holding on to the former high active lipids formerly associated with PC. Most diseases and aging tends towards a higher concentration of raft formation. This is complicated with signaling emanating from rafts that encourages apoptosis, which is both destructive and constructive.

The low activity level of the three lipids encourages the agglomeration into rafts which ultimately degrades the fluidity of vibrant active membranes. Most diseases and aging tend towards a higher concentration of raft formation. This is complicated with signaling emanating from rafts that encourage apoptosis, which is both destructive and constructive.

Although scientists have long linked butyrate to overall reductions in the incidence of colon cancer, the molecular basis of that benefit has remained largely unknown. Butyrate affects a chemical that otherwise binds and constricts the activity of the p21 gene that is involved in the growth of cancer cells. Butyrate optimizes itself in the body. Concentrations of butyrate in the composition of the invention can range from about 1-10 grams per liter or more, depending on the specific condition at hand. Minamiyama et al. Hum. Mol. Genet. 1:13(11):1183-92 (2004), (incorporated herein by reference by its entirety) in a study using mouse model of Bulbar ALS, demonstrated oral administration of sodium butyrate (SB) successfully ameliorated neurological phenotypes as well as increased acetylation of nuclear histone in neural tissues.

When β-oxidation of Renegade fatty acids is impaired, sodium phenylbutyrate (PBA) is used that is a short chain fatty acid and has a long clinical history of treatment for hyperammonemia and urea cycle disorders (ornithine transcarbamylase deficiency) without adverse effects. The use of sodium phenylbutyrate or calcium/magnesium butyrate, a short 4-carbon chain fatty acid, is of striking benefit in breaking apart and mobilizing renegade fats, lowering glutamate and aspartate, affecting neuronal excitability, sequestering ammonia, clearing biotoxins, preventing cerebral ischemic injury, acting as a histone deacetylase inhibitor as well as having neuroprotective effects.

In ALS, Alzheimer's, Post Stroke and ASD models PBA addresses the formation of lipid rafts, and neuroinflammation as well as having neuroprotective effects as a histone deacetylase inhibitor and prolonging survival and regulating expression of anti-apoptotic genes. PBA inhibits the induction of iNOS (inducible nitric oxide synthase) and proinflammatory cytokines such as tumor necrosis factor alpha in astrocytes, microglia and macrophages implicating a neuroprotective role. PBA has also been shown to suppress the proliferation of myelin basic protein primed T cells and may inhibit the disease process of experimental allergic encephalomyelitis.

In one embodiment of the invention, there is provided treatment methods and compositions containing PBA. The adult patients with ALS have demonstrated marked positive responses to intravenous use of sodium phenylbutyrate. The pediatric patients have used both the IV sodium phenylbutyrate and oral phenylbutyrate (e.g., about 1 gram to about 6 grams IV) for several years with a dosage of 1, 2, 3, 4, 5, or 6 grams daily. Prior to the introduction of phenylbutyrate, membrane lipid stabilization must be achieved with essential fatty acids and phosphatidylcholine. The aggressive use of IV sodium phenylbutyrate without essential fatty acids and PC leads to clinical instability in adult patients with ALS.

2.6 Electrolytes

Electrolyte is a “medical/scientific” term for salts, specifically ions. The term electrolyte means that ion is electrically-charged and moves to either a negative (cathode) or positive (anode) electrode. Electrolytes are vital elements of a healthy body and are needed for the proper performance of bodily organs and tissues by maintaining the voltages across the cell membranes and to carry electrical impulses (nerve impulses, muscle contractions) across these cells and to other cells. The kidneys function is to keep the electrolyte concentrations constant in the blood despite changes in the body. For example, during a heavy exercise the body loses electrolytes in the sweat, particularly sodium and potassium. These electrolytes must be replaced to keep the electrolyte concentrations of the body fluids constant. So, many sports drinks have sodium chloride or potassium chloride added therein.

The types of electrolytes used within the scope of the invention include, by way of example and not limitation, sodium (Na⁺), potassium (K⁺), chloride (Cl⁻), Calcium(Ca²), Magnesium (mg²), bicarbonate (HCO₃⁻), Phosphate (PO₄⁻²) and sulfate (SO₄⁻²), among others.

2.7 Trace Minerals

Suitable mineral compositions that may optionally be included in the PK Protocol include solid multi-mineral preparations, or the E-Lyte Liquid Mineral™ set #1-8 (separate solutions of biologically available potassium, zinc, magnesium, copper, chromium, manganese, molybdenum, selenium and iodine, or a combination thereof, or #1-9 (separate solutions of biologically available potassium, zinc, magnesium, copper, chromium, manganese, molybdenum, selenium and iodine), or a combination thereof. Both E-Lyte Liquid Mineral™ set #1-8, and E-Lyte Liquid Mineral™ set #1-9 set are available from E-Lyte, Inc. (Millville, N.J., USA).

### 3. Formulation and Mode of Administration

The active compositions of the invention having tissue modulatory activities as described herein are provided as isolated and substantially purified compounds in pharmaceutically acceptable formulations using formulation methods known to those of ordinary skill in the art. These formulations can be administered by standard routes.

In a specific embodiment, the term “pharmaceutically acceptable” means approved by a regulatory agency of the Federal or a state government or listed in the U.S. Pharmacopoeia or other generally recognized pharmacopoeia for use in animals, and more particularly in humans.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Preferred oil is essential fatty acids, linoleic acid and linolenic acid. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In general, the combinations may be administered by the transdermal, intraperitoneal, intracranial, intracerebroventricular, intracerebral, intravaginal, intrauterine, oral, rectal, ophthalmic (including intravitreal or intracameral), nasal, topical (including buccal and sublingual), parenteral (including subcutaneous, intraperitoneal, intramuscular, intravenous, intradermal, intracranial, intratracheal, and epidural and nasal) administration. Parenteral administration includes direct or indirect injection into cells, tissues or organs in vivo, ex vivo or in vitro.

Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubelizing agent and a local anesthetic such as procaine to ease discomfort at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided. The compositions are administered separately or are mixed together prior to administration.

In one embodiment, the first composition, the second composition or both may be incorporated into biodegradable polymers allowing for sustained release of the compound, the polymers being implanted in the vicinity of where drug delivery is desired, for example, at the site of a spinal cord injury or implanted so that the composition is slowly released systemically. Osmotic mini-pumps may also be used to provide controlled delivery of the first composition, the second composition or both through cannulae to the site of interest, such as directly into the site of injury. The biodegradable polymers and their use are described, for example, in detail in Brem et al., J. Neurosurg. 74:441-446 (1991), which is hereby incorporated by reference in its entirety.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

The composition formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

In one embodiment of the invention, the composition is prepared for topical administration in saline (combined with any of the preservatives and antimicrobial agents commonly used in ocular preparations), and administered in eye drop form. The solution or suspension may be prepared in its pure form and administered several times daily. Alternatively, the pharmaceutical composition, prepared as described above, may also be administered directly to the cornea. Within preferred embodiments, the composition is prepared with a muco-adhesive polymer which binds to cornea. Within further embodiments, the antiangiogenic factors or antiangiogenic compositions may be utilized as an adjunct to the combination therapy.

In another embodiment, the composition of the invention comprises a therapeutically effective amount of a first composition comprising one or more phosphatidylcholine formulations and the second composition comprising EPO, and optionally one or more constituents comprising essential fatty acid supplements, trace minerals, butyrate, electrolytes, methylating agents (methylcobalamin, folinic acid/Leucovorin), glutathione, or a combination thereof, in a suitable carrier.

A typical regimen for treatment of symptoms of diseases and disorders related to impaired development and activities of cells and tissues comprises administration of an effective amount of the composition as described above, administered as a single treatment, or repeated as enhancing or booster dosages, over a period up to and including one week to about 48 months or more.

Within other embodiments, the compositions may also be placed in any location such that the compounds or constituents are continuously released into the aqueous humor. The amount of the composition of the invention which will be effective in the treatment of symptoms of diseases and disorders related to impaired tissue development can be determined by standard clinical techniques. In addition, in vitro assays may optionally be employed to help identify optimal dosage ranges. In particular, the dosage of the compositions of the present invention will depend on the disease state of subject under treatment and other clinical factors such as weight and condition of the human or animal and the route of administration of the compounds or compositions. The precise dose to be employed in the formulation, therefore, should be decided according to the judgment of the health care practitioner and each patient's circumstances. Effective doses may be extrapolated from dose-response curves derived from in vitro or animal model test systems.

Within another embodiment of the present invention, methods are provided for treating or preventing neovascular glaucoma, comprising the step of administering to a patient a therapeutically effective amount of the compositions of the invention to the eye, such that the formation of blood vessels is modulated. In one embodiment, the compound may be administered topically to the eye in order to modulate early forms of neovascular glaucoma.

Within other embodiments, the composition may be implanted by injection into the region of the anterior chamber angle. Within other embodiments, the composition may also be placed in any location such that the composition is continuously released into the aqueous humor.

Within one aspect of the present invention, methods are provided for treating or preventing proliferative diabetic retinopathy, comprising the step of administering to a patient the combination therapy of the invention in a therapeutically effective amount such that the formation of blood vessels is modulated.

Within another aspect of the present invention, methods are provided for treating or preventing retrolental fibroplasia, comprising the step of administering to a patient a therapeutically effective amount of the composition of the invention to the eye, such that the formation of blood vessels is modulated. The compounds may be administered topically, via intravitreous injection and/or via intraocular implants.

Within one aspect of the present invention, the pharmaceutical composition of the invention may be administered to the resection margin of a wide variety of tumors, including for example, breast, colon, brain and hepatic tumors. For example, within one embodiment of the invention, the compositions may be administered to the site of a neurological tumor subsequent to excision, such that the formation of new blood vessels at the site is modulated.

Various delivery systems are known and can be used to administer a compound of the invention, i.e., encapsulation in liposomes, microparticles, microcapsules, recombinant cells capable of expressing the compound, receptor-mediated endocytosis (see, i.e., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (i.e., oral mucosa, rectal and intestinal mucosa, etc.) and may be administered together with other biologically active agents. Administration can be systemic or local.

In addition, it may be desirable to introduce the compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, i.e., by use of an inhaler or nebulizer, and formulation with an aerosolizing agent.

In a specific embodiment, it may be desirable to administer the compositions of the invention locally to the area in need of treatment; this may be achieved by, for example, and not by way of limitation, local infusion during surgery, topical application, i.e., in conjunction with a wound dressing after surgery, by injection, by means of a catheter, by means of a suppository, or by means of an implant, the implant being of a porous, non-porous, or gelatinous material, including membranes, such as sialastic membranes, or fibers. Preferably, when administering a protein such as EPO, care must be taken to use materials to which the protein does not absorb or otherwise interact.

In one embodiment, the compound or composition can be delivered in a controlled release system. In one embodiment, a pump may be used (see, Sefton, Biomed. Eng. 14:201 (1987)). In another embodiment, polymeric materials can be used (see, Ranger and Peppas, J., Macromol. Sci. Rev. Macromol. Chem. 23:61 (1983); and Levy et al., Science 228:190 (1985)). In yet another embodiment, a controlled release system can be placed in proximity of the therapeutic target, i.e., the brain, thus requiring only a fraction of the systemic dose. Other controlled release systems are discussed in the review by Langer, Science 249:1527-1533 (1990).

Treating humans or animals between approximately 0.5 to 500 mg/kilogram is a typical broad range for administering the composition of the invention. The methods of the present invention contemplate single as well as multiple administrations, given either simultaneously or over an extended period of time.

Preferred unit dosage formulations are those containing a daily dose or unit, daily sub-dose, or an appropriate fraction thereof, of the administered compositions. It should be understood that in addition to the compositions, particularly mentioned above, the formulations of the present invention may include other agents conventional in the art having regard to the type of formulation in question.

The composition of the invention comprises a dry formulation, an aqueous solution, or both. Effective amounts of a phosphatidylcholine composition and EPO can be formulated into one or two solutions for treating impairment of tissue regeneration related disease or disorders, such as for example, CNS related diseases, and for delaying the onset of these disease symptoms in a subject. The compositions for parenteral administration are characterized as being sterile and pyrogen-free. One skilled in the art can readily prepare the composition for combination therapy of the invention for enteral or parenteral use, for example by using the principles set forth in Remington's Pharmaceutical Science, 18^{th} edit. (Alphonso Gennaro, ed.) Mack Publishing Co., Easton, Pa., 1990.

Because phosphatidylcholine, linoleic acid and alpha linolenic acid are all soluble in oils or lipids, they can be conveniently formulated into a single composition. Thus, in one embodiment, the invention provides a single-dose composition comprising a phosphatidylcholine composition and an EFA 4:1 composition (PC 4:1).

The compositions of the invention can be in a form suitable for oral use, according to any technique suitable for the manufacture of oral pharmaceutical compositions as are within the skill in the art. For example, the phosphatidylcholine composition and the EFA composition can be formulated (either separately or together) into soft capsules, oily suspensions, or emulsions, optionally in admixture with pharmaceutically acceptable excipients. Suitable excipients for a phosphatidylcholine composition or EFA composition comprise oil-based media; e.g., arachis oil, liquid paraffin, or vegetable oils such as olive oil. Butyrate is administered in encapsulated form, for example, as Magnesium/Calcium Butyrate from BodyBio, Inc., (Millville, N.J., USA) or Sodium Phenylbutyrate from Triple Crown America (Perkasie, Pa., USA) or as IV Liquid Sodium PhenylButyrate from Wellness Health and Pharmaceuticals (Birmingham, Ala., USA).

The compositions of the invention are formulated into liquid or solid compositions, such as aqueous solutions, aqueous or oily suspensions, syrups or elixirs, emulsions, tablets, dispersible powders or granules, hard or soft capsules, optionally in admixture with pharmaceutically acceptable excipients.

2.1. Adjuvants, Carriers, and Diluents

As would be understood by one of ordinary skill in the art, when a composition of the present invention is provided to an individual, it can further comprise at least one of salts, buffers, adjuvants, or other substances which are desirable for improving the efficacy of the composition. Adjuvants are substances that can be used to specifically augment at least one immune response. Normally, the adjuvant and the composition are mixed prior to presentation to the immune system, or presented separately.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

Adjuvants can be generally divided into several groups based upon their composition. These groups include lipid micelles, oil adjuvants, mineral salts (for example, AlK(SO₄)₂, AlNa (SO₄)₂, AlNH₄ (SO₄)), silica, kaolin, and certain natural substances, for example, wax D from Mycobacterium tuberculosis, substances found in Corynebacterium parvum, or Bordetella pertussis, Freund's adjuvant (DIFCO), alum adjuvant (Alhydrogel), MF-50 (Chiron) Novasomes™, or micelles, among others.

Suitable excipients for liquid formulation include water or saline, suspending agents such as sodium carboxymethylcellulose, methylcellulose, hydroxypropylmethyl-cellulose, sodium alginate, polyvinylpyrrolidone, gum tragacanth, and gum acacia; dispersing or wetting agents such as lecithin, condensation products of an alkylene oxide with fatty acids (e.g., polyoxethylene stearate), condensation products of ethylene oxide with long chain aliphatic alcohols (e.g., heptadecethyleneoxy-cetanol), condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyoxyethylene sorbitan monooleate).

Suitable excipients for solid formulations include calcium carbonate, sodium carbonate, lactose, calcium phosphate, or sodium phosphate; granulating and disintegrating agents such as maize starch, or alginic acid; binding agents such as starch, gelatin, or acacia; and lubricating agents such as magnesium stearate, stearic acids, or talc, and inert solid diluents such as calcium carbonate, calcium phosphate, or kaolin.

Other suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides.

Oral compositions of the invention can contain one or more agents selected from the group consisting of sweetening agents, flavoring agents, coloring agents, and preserving agents in order to provide a pharmaceutically palatable preparation.

Liquid formulations according to the invention can contain one or more preservatives such as ethyl, n-propyl, or p-hydroxy benzoate; one or more coloring agents; one or more flavoring agents; or one or more sweetening agents such as sucrose, saccharin, or sodium or calcium cyclamate.

Liquid formulations according to the invention, especially those comprising a phosphotidylcholine composition and/or an EFA composition can contain antioxidants such as tocopherol, sodium metabisulphite, butylated hydroxytoluene (BHT), butylated hydroxyanisole (BHA), ascorbic acid or sodium ascorbate.

The amount of the compound of the invention which will be effective in the treatment, inhibition and prevention of a disease or disorder associated with tissue or cell impairment can be determined by standard clinical techniques. In addition, in vitro assays may optionally be employed to help identify optimal dosage ranges.

In particular, the dosage of the composition of the present invention will depend on the disease state or condition being treated and other clinical factors such as weight and condition of the human or animal and the route of administration of the compound. The precise dose to be employed in the formulation, therefore, should be decided according to the judgment of the practitioner and each patient's circumstances. Effective doses may be extrapolated from dose-response curves derived from in vitro or animal model test systems.

For treating humans or animals, between approximately 0.5 to 500 mg/kilogram, is a typical broad range for administering the pharmaceutical composition of the invention. The methods of the present invention contemplate single as well as multiple administrations, given either simultaneously or over an extended period of time. It is to be understood that the present invention has application for both human and veterinary use.

Preferred unit dosage formulations are those containing a daily dose or unit, daily sub-dose, as herein above recited, or an appropriate fraction thereof, of the administered ingredient. It should be understood that in addition to the ingredients, particularly mentioned above, the formulations of the present invention may include other agents conventional in the art having regard to the type of formulation in question.

The invention also provides a combination therapy pack or kit comprising one or more containers filled with one or more of the ingredients of the PK Protocol and EPO compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### 3. Methods of Treating CNS-related Diseases

A subject presenting with symptoms indicative of a CNS-related disease can be treated by the methods and compositions of the invention to prevent, delay onset, ameliorate and/or treat one or more symptoms of CNS-related symptoms. The “treatment ” provided need not be absolute, i.e., CNS related disease need not be totally prevented or treated, provided that there is a statistically significant improvement relative to a control population. Treatment can be limited to mitigating the severity or rapidity of onset of symptoms of the disease.

A typical regimen for preventing, suppressing, or treating a disease or condition related to CNS diseases comprises administration of an effective amount of the composition as described above, administered as a single treatment, or repeated as enhancing or booster dosages, over a period up to and including one week to about 48 months or more, or permanently if it need be.

An “effective amount” of the compositions of the invention is any amount sufficient to therapeutically inhibit the progression of symptoms of diseases and disorders related to impaired development and activities of cells and tissues or to pyrophylactically delay the onset of impaired development of tissue.

One of ordinary skill in the art can readily determine an appropriate temporal and interval regimen for administering the compositions of the invention. For example, the compositions of the invention can be administered once, twice or more daily, for one, two, three, four, five, six or seven days in a given a week, for one or several weeks or months. The length of time that the subject receives the composition can be determined by the subject's physician or other health care providers and caretakers, according to need. Due to the chronic and progressive nature of CNS-related disease, it is expected that subjects will receive one or more compositions according to the present methods for a definite or an indefinite period of time.

In one embodiment of the invention, a phosphatidylcholine composition containing about 1500 mg to 2000 mg phosphatidylcholine is administered to a subject intravenously, for example one to two times daily, for consecutive or non-consecutive days in a given week. Another phosphatidylcholine composition which contains about 3600 mg to about 18,000 mg phosphatidylcholine is administered, for example once or twice, to the same subject daily by mouth.

In another embodiment, one or more compositions comprising EPO, PC and linoleic acid and alpha linolenic acid in an approximately 4:1 (v/v) ratio are administered to a subject who has been diagnosed with, or has demonstrated one or more symptoms of spinal stenosis, ALS, Multiple Sclerosis, Autism, Post Stroke, Parkinson's or Alzheimer's disease. Linoleic acid, and alpha linolenic acid, can be administered separately to a subject, as long as the ratio (v/v) of linoleic acid to alpha linolenic acid administered within a given time frame (e.g., 24 hours or less, 12 hours or less, 6 hours or less, or 4 hours or less) is approximately 4:1. The term “EFA 4:1 composition” therefore refers to one or more compositions comprising linoleic acid and one or more compositions comprising alpha linolenic acid, which are administered separately or together to a subject at about 4:1 (v/v) ratio of linoleic acid to alpha linoleic acid.

Any commercially available preparation comprising linoleic acid and alpha linolenic acid, or mixtures of the two in an approximately 4:1 (v/v) ratio, can be used as the EFA 4:1 composition in the present methods. Suitable EFA 4:1 compositions include the BodyBio Balance 4:1™ EFA oil available from BodyBio Inc. (Millville, N.J. USA), or any mixtures containing the essential fatty acids, such as for example, a mixture of cold pressed organic safflower or sunflower oil and flaxseed oil to yield a 4:1 ratio of linoleic acid to linolenic acid (4 parts Omega 6: to 1 part Omega 3).

The EFA compositions can be administered to a subject by any parenteral or enteral technique suitable for introducing the EFA composition into blood stream or the gastrointestinal tract. In a preferred embodiment, the EFA 4:1 compositions are administered to the subject by mouth. For example, an effective amount of the EFA 4:1 composition can be from about 10 mls (about 2 teaspoons) to about 100 mls (about 7 tablespoons), about 15 mls (about 1 tablespoon) to about 80 mls (about 5 tablespoons), or about 30 mls (about 2 tablespoons) to about 60 mls (about 4 tablespoons). In one embodiment, about 30 mls to about 60 mls (about 2 to about 4 tablespoons) of the EFA 4:1 composition is administered to a subject by mouth, once to twice daily. In another embodiment, gamma linolenic acid is administered by mouth as evening primrose oil from about 910 mg to about 3960 mg.

An “effective amount” of the EPO compositions is any amount sufficient to inhibit the progression of spinal stenosis, or to delay the onset of spinal stenosis symptoms, when administered in conjunction with the phosphatidylcholine and one or more compositions containing trace minerals, rGlutathione, butyrate, electrolytes, methylating agents (folinic acid, methylcobalamin), or a combination thereof. For example, an effective amount of the EPO composition can be from about 5000 units to 20,000 units, one or two times per week.

One skilled in the art can readily determine an appropriate dosage regimen for administering the compositions of the invention. For example, the compositions of the invention can be administered once, twice or more daily, for one, two, three, four, five, six or seven days in a given week. The length of time that the subject receives the compositions can be determined by the subject's physician according to need. According to the severity of the symptoms of CNS-related disease and its chronic or progressive nature, subjects may be expected to receive the compositions according to the present methods for weeks or months.

In the practice of the present methods, an effective amount of compositions comprising trace minerals are administered to subject who has been diagnosed with, or who is at risk for developing autism. The trace minerals in one or more same or different compositions are administered to the subject, or two or more mineral compositions can be administered separately. It is understood that mineral compositions can be administered separately to a subject, as long as the compositions are administered within a given time frame (e.g., 24 hours or less, preferably 12 hours or less, more preferably 6 hours or less, particularly preferably 4 hours or less). Preferably, mineral compositions for use in the present methods comprise biologically available forms of potassium, magnesium, zinc, copper, chromium, manganese, molybdenum, selenium, iodine, or any combination thereof, although the mineral compositions can comprise other minerals in biologically available form.

The compositions comprising trace minerals can be administered to a subject by any parenteral or enteral technique suitable for introducing the compositions into the blood stream or gastrointestinal tract. In one embodiment, the compositions comprising trace minerals are administered to the subject by mouth.

Also encompassed within the scope of the invention is the use of the electrolytes. In one embodiment, a balanced electrolyte concentrate is administered orally with one to fifteen tablespoons diluted in fluid. E-Lyte Balanced Electrolyte is a concentrated high K:Na ratio solution that is usually diluted with H₂O at 16:1. In another embodiment the subject is instructed to take the electrolyte in its concentrated form, one to three tablespoons at a time followed by 1 or 2 ounces of H20, throughout the day.

Any commercially available composition or compositions comprising one or more biologically available minerals can be used as trace mineral composition of the present invention. Suitable mineral compositions include solid multi-mineral preparations, or the E-Lyte Liquid Mineral™ set #1-8 (separate solutions of biologically available potassium, zinc, magnesium, copper, chromium, manganese, molybdenum, and selenium) or #1-9 (separate solutions of biologically available potassium, zinc, magnesium, copper, chromium, manganese, molybdenum, selenium and iodine), both available from E-Lyte, Inc. (Millville, N.J. USA). The effective amount of the trace minerals is determined for each subject according to that subject's needs and disease status and evaluation.

After determining the effective amount of the one or more mineral compositions for administration to the subject, one skilled in the art can readily determine the dosage regimen for administering mineral compositions. For example, the trace minerals can be administered once, twice or more daily, for one, two, three, four, five, six or seven days in a given week. Preferably, the one or more mineral compositions are administered to the subject twice a day, for seven days in a given week. The length of time that the subject receives the mineral compositions can be determined by the subject's physician or primary caretaker, according to need.

In another embodiment, a subject being treated according to the present methods receives intravascular (e.g., intravenous) reduced Glutathione. For example, a subject can receive from about 1000 mg to about 3000 mg of rGlutathione, about 1500 mg to about 2800 mg rGlutathione, about 1800 mg to about 2400 mg rGlutathione, once, twice or more daily, for one, two, three, four, five, six or seven days a week. In one embodiment, the subject receives about 1800 mg to about 2400 mg intravenous rGlutathione twice daily, for three consecutive or non-consecutive days in a given week. In another embodiment, the rGlutathione is administered in reduced form as an intravenous “fast push” over three to five minutes.

Any commercially available composition comprising rGlutathione can be used in the present methods. Suitable compositions comprising rGlutathione include the rGlutathione preparations from Wellness Pharmacy, Inc. (Birmingham, Ala., USA) (has patented method of stabilization of glutathione with two patents, U.S. Pat. No. 7,449.546 and U.S. Pat. No. 6,835,811), or Tationil (Roche, Italy).

It is also preferable to maintain a subject being treated by the present methods on a low carbohydrate, high protein, high green vegetable, high legume as butter beans/mucuna, high fat diet termed the Detoxx Diet, e.g., a diet excluding all grains, sugars, fruit, fruit juices, all “below ground” root vegetables and processed foods. Suitable low carbohydrate, high protein, high fat diets include such well-known diets as Atkins® or the South Beach Diet™ (see, e.g., Atkins RC, Atkins for Life, St. Martins Press, NY, 2003 and Agatston A, THE SOUTH BEACH DIET: THE DELICIOUS, DOCTOR-DESIGNED, FOOLPROOF PLAN FOR FAST AND HEALTHY WEIGHT Loss, Random House, NY, 2003, the entire disclosures of which are herein incorporated by reference). A diet lower in carbohydrate suppresses phospholipase A2 (PLA2), an enzyme that stimulates the catalyzing or breaking apart of the essential fatty acids from the phospholipids in the cell membrane, thereby de-stabilizing the membrane and control of cellular function.

Oral support with neurotransmitter precursors is helpful with the amino acids tryptophan, 5-hydroxytryptophan, theonine, mucuna beans, butter beans, tyrosine, and phenylalanine as indicated by testing of urinary neurotransmitters.

In one embodiment, the subject being treated for a CNS-related disease receives EPO and rGlutathione as well as phosphatidylcholine and Leucovorin, which are administered intravenously and methylcobalamin is administered by injection.

In another embodiment, the present methods comprise treating a subject who has been diagnosed with a CNS-related disease, or who is at risk for developing one or more symptoms of CNS-related disease, for a definite period of time (e.g., five weeks or more) by:

1) intravenous administration by lipid exchange of a phosphatidylcholine (PC) composition comprising about 1500 mg phosphatidylcholine (e.g., bolus PC of 2 to 5 grams), followed by intravenous administration of EPO at about 10,000 Units that may or may not be mixed with 250 mg PC, followed by second administration of PC in an amount of about 1500 mg.

### 5. Test Kits

The invention also provides a combination therapy pack or kit comprising one or more containers filled with one or more compositions or the ingredients of the combined therapy of the invention. The kits are provided for the treatment of the symptoms of disease and disorders related to impaired development and activities of cells and tissues. The kit comprises instructions for treating the disease or disorder in a subject and two active compositions, Kane composition and the growth factor composition. The Kane compostion includes one or more of the following components: 1) a phosphatidylcholine composition; 2) an EFA 4:1 composition; 3) mineral compositions, 4) electrolyte compositions; 5) methylating agents, methylcobalamin and folinic acid/Leucovorin; 6) rGlutathione; 7) butyrate or phenylbutyrate, or a combination thereof. The growth factor composition contains a growth factor, cytokine or growth hormone. In one embodiment, the growth factor is EPO.

If a particular component is not included in the kit, the kit can optionally comprise information on where to obtain the missing component, for example an order form or uniform resource locator for the internet specifying a website where the component can be obtained. The instructions provided with the kit describe the practice of the methods of the invention as described above, and the route of administration and effective concentration and the dosing regimen for each of the compositions provided therein.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims. The contents of all references, patents and published patent applications cited throughout this application are expressly incorporated herein by reference.

### EXAMPLES

### Example 1

### Preparation of BodyBio PC

Bodybio PC was prepared from a concentrated phosphatidylcholine that contained triple lecithin as a starting material. The starting material is a byproduct of soy oil manufactured from Central Soya or Archers, Daniel, Midland (ADM). The starting phosphotidylcholine material was further concentrated in order to increase the concentration of phospholipids and reduce the concentration of the triglyceride therein. The process of concentration was performed by Liposome Labs, at 301 N 1st Street East, Snowflake, Ariz. 85937, which in the process includes the addition of ethanol with the subsequent requirement for vacuum distillation to remove the majority of the alcohol. As a result of the concentration process an intermediate phosphatidylcholine compound was generated that was a cake-like substance containing high level of phospholipids and a favorable ratio of omega 6 and omega 3 fatty acids. The fatty acids and phospholipid concentration in the intermediate phosphatidylcholine compound is presented below:

### Fatty Acid Content:

C_{16.0} 16.1%
C_{16.1} 0.1%
C_{18.0} 4.1%
C_{18.1} 10.0%
C_{18.2} 55.30% (omega 6)
C_{18.3} 14.0% (omega 3)
C_{22.0} 0.4%

### Phospholipids:

Phosphatidylcholine: 50%
Phosphatidylethanolamine 14.4%
Phosphatidylinositol 2.4%

The intermediate phosphatidylcholine compound was then subjected to a liquidation process. The liquidation process was achieved by adding flax seed oil to the vacuum distilled phosphatidylcholine intermediate compound. The flax seed oil was added in an amount of approx. 9% v/w, which resulted in the end product of phosphatidylcholine (BodyBio PC). BodyBio PC contains a ratio of about 4 parts linoleic acid to about 1 part alpha linolenic acid. The end product was then subjected to the process of bottling and/or encapsulation.

### Example 2

### Treatment of Symptoms Related to CNS Related Disorder with IV Combination Therapy

The case studies represented below represented the result of the treatment regimen on subjects suffering from CNS-related disorders. Phospholipid re-modeling of these subjects was stimulated by supplying IV phospholipids, principally BodyBio PC and/or Lipostabil phoshaptidylcholine. Erythropoeitin composition was PROCRIT®. (Epoetin alfa) Manufactured by: Amgen Inc. One Amgen Center Drive, Thousand Oaks, Calif. 91320-1789, Distributed by: Ortho Biotech Products, L.P. Raritan, New Jersey 08869-0670. Mode of Administration was one intravenous infusion weekly or bi-weekly. Dosage of Compound was 10,000 units per week or bimonthly with pre and post administration of 1500 mg of Lipostabil phoshaptidylcholine.

Patients with nerve damage due to spinal stenosis were previously examined and were recorded as being unable to walk without losing balance and required the use of a cane. Patients received weekly infusions of EPO with pre and post administration of Lipostabil phoshaptidylcholine. After 3 weeks patients no longer required a cane for stability. After 5 infusions patients were able to walk 1-1.5 mile daily. After 7 infusions walking was increased to 2 miles daily. After 9 infusions patients were able to walk for more than 2 miles daily. After 9 weeks of the combination therapy most patients demonstrated an improvement in hearing, memory, cognition, productivity and energy as well gait and balance.

### Example 3

### Intravenous Administration of PK Protocol and EPO Compositions

a) Administration of Sodium Phenylbutyrate (PB)

A butterfly catheter with a 23-gauge needle was inserted into a vein of the antecubital region of one of the subjects' arms. A syringe containing a Sodium Phenylbutyrate composition of 1 to 2 grams of phenylbutyrate diluted in 50 to 55 ml of D5W (5% dextrose in water) was connected to the catheter by a flexible tube. The PB composition was then infused (or “pushed”) into the subject's vein over a period of 15 to 20 minutes. Alternatively, 5 to 10 grams of sodium phenylbutyrate may be slowly dripped intravenously by dilution in 250 ml of D5W over 1 to 2 hours.

b) Administration of PC Composition (first)

A syringe containing the PC (phosphatidylcholine) composition of about 30 cc volume diluted with 30 cc D5W (5% dextrose in water) was connected to the catheter by a flexible tube. The PC composition was then infused (or “pushed”) into the subject over a period of ten minutes.

c) Infusion is continued with the administration of EPO 10,000 Units mixed with 250 mg of PC

d) Administration of PC Composition (second) in the next step of intravenous administration another 30 cc volume diluted with 30 cc D5W (5% dextrose in water) was connected to the catheter by a flexible tube. The PC composition was then infused (or “pushed”) into the subject's vein over a period of ten minutes.

e) Another step in the infusion continues the process with the B vitamin, Leucovorin (Folinic Acid) in a pre-prepared syringe containing about 2 mg (0.2 cc) to about 10 mg (1 cc) of Leucovorin over the period of 2-3 minutes.

f) Intravenous Administration of Reduced Glutathione

The PB, PC (1^{st}), EPO, PC (2^{nd}) and Leucovorin compositions were infused first followed by a pre-prepared syringe containing about 1.5 to 20 cc (300 to 4000 mg) of glutathione generally pre-mixed with an equal portion of sterile water (not saline).

This procedure avoids numerously piercing of the patient by infusing first the PB, then the first PC, then the EPO, then the second PC, the Leucovorin and then the glutathione using the same butterfly catheter with a flexible tube infused (or “pushed”) into the subject over a period of 45 minutes to one hour.

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

What is claimed is:

1. A method of preventing, ameliorating, or treating one or more symptoms of diseases related to impairment of development and activities of cells and tissues in a subject in need of treatment thereof, comprising administration of a combination therapy comprising at least two active compositions, a first active composition comprising a balanced phosphatidylcholine formulation and an essential fatty acid composition, and a second active composition comprising a growth factor, in a suitable carrier or diluent, wherein the combination therapy modulates development and activities of cells and tissues in the subject.

2. The method of claim 1, wherein the tissue comprises epithelial tissue, connective tissue, muscle tissue, nerve tissue, or a combination thereof.

3. The method of claim 2, wherein the tissue comprises nerve tissue and the therapy is used for prevention, treatment, and/or amelioration of one or more symptoms of a neurodegenerative disease.

4. The method of claim 3, wherein the neurodegenerative disease comprises spinal stenosis, ALS, Multiple Sclerosis, Autism, Post Stroke, Parkinson's and Alzheimer's disease.

5. The method of claim 1, wherein the growth factor comprises erythropoietin.

6. The method of claim 5, wherein the erythropoietin comprises comprises natural erythropoietin, a recombinant erythropoietin, a recombinant epoetin alfa, or a combination thereof.

7. The method of claim 1, wherein the first active composition and the second active composition are administered successively, contemporaneously, or both.

8. The method of claim 1, wherein the first active composition and the second active composition are formulated in one solution.

9. The method of claim 1, wherein the first active composition and the second active composition are formulated in different solutions.

10. The method of claim 1, wherein the first active composition, the second active composition, or both are administered at different time intervals.

11. The method of claim 1, wherein the first active composition, the second active composition, or both are administered in a time-released manner.

12. The method of claim 1, wherein the first active composition, the second active composition, or both are in a dry formulation, liquid formulation, or both.

13. The method of claim 1, wherein the first active composition is administered at a dosage of about 50 units/kg, 100 Units/kg or 150 Units/kg at a weekly or biweekly interval.

14. The method of claim 13, wherein the first active composition, the second active composition, or both are administered parenteraly.

15. The method of claim 14, wherein the first active composition, the second active composition, or both are administered intravenously.

16. The method of claim 15, comprising one intravenous infusion weekly or bimonthly in orderly steps of:
i) intravenous administration of about 1500 mg of the balanced PC;
ii) intravenous administration of about 10,000 unit of recombinant epoetin alfa; and
iii) intravenous administration of about 1500 mg of the balanced PC,
wherein a modulation of development and activities of cells and tissues in the subject is commenced after one or more intravenous infusions.

17. A composition for preventing, ameliorating and/or treating symptoms of diseases related to impairment of development and activities of cells and tissues comprising at least two active ingredients, a first active ingredient comprising erythropoietin and a second active ingredient comprising a balanced PC, in a suitable carrier or diluent.

18. The composition of claim 17 further comprising one or more constituents comprising linoleic acid and alpha linolenic acid in a ratio of about 4:1, trace minerals, butyrate or phenylbutyrate, electrolytes, methylating agents, glutathione, or a combination thereof.

19. The composition of claim 17, wherein the balanced PC is BodyBio PC, LipoStabil N, Essentiale N, or a combination thereof and erythropoietin is a recombinant erythropoietin.

20. A kit for modulation of development and activities of cells and tissues in a subject, comprising:
a) a first active composition comprising one or more erythropoietin formulations;
b) a second active composition comprising a balanced PC; and
c) optionally containing additional one or more constituents comprising:
i) linoleic acid and alpha linolenic acid in a ratio of about 4:1;
ii) trace minerals;
iii) butyrate or phenylbutyrate;
iv) electrolytes;
v) methylating agents; and
vi) glutathione;
d) instructions for the use of the first and second active compositions and the constituents; and
e) instructions for where to obtain any missing components of the kit.
